(19) United States
(12) Patent Application Publication
(10) Pub. No.: US 2022/0327952 A1
(43) Pub. Date: Oct. 13, 2022

(54) INTERACTION MONITORING SYSTEM, PARENTING ASSISTANCE SYSTEM USING THE SAME AND INTERACTION MONITORING METHOD USING THE SAME

(71) Applicant: Korea Advanced Institute of Science and Technology, Daejeon (KR)

(72) Inventors: Junehwa Song, Daejeon (KR); Wonjung Kim, Daejeon (KR); Seungchul Lee, Daejeon (KR); Seonghoon Kim, Daejeon (KR); Sungbin Jo, Daejeon (KR); Chungkuk Yoo, Daejeon (KR); Inseok Hwang, Pohang (KR)

(73) Assignee: Korea Advanced Institute of Science and Technology, Daejeon (KR)

(21) Appl. No.: 17/555,457

(22) Filed: Dec. 19, 2021

(30) Foreign Application Priority Data
Apr. 7, 2021 (KR) 10-2021-0045326

(51) Int. Cl. G09B 19/00; A61B 5/0531; A61B 5/024; A61B 5/00; A61B 5/16; A61B 5/0205; G06V 40/20; G06V 40/16

(52) U.S. Cl. CPC G09B 19/00; A61B 5/0531; A61B 5/024; A61B 5/4803; A61B 5/163; A61B 5/165; A61B 5/749; A61B 5/742; A61B 5/0002; A61B 5/0077; A61B 5/0205; G06V 40/20; G06V 40/161

(57) ABSTRACT

Disclosed herein is an interaction monitoring system, comprising an environment collection module, interaction monitoring module, interaction segmentation module, and display module. The environment collection module detects surrounding environment and generates a data stream. The interaction monitoring module generates a feature data stream by extracting feature value of the data stream. The interaction segmentation module determines a target situation, which indicates a user's state or condition, from the feature data stream and generates a target image or video stream, which indicates the target situation. The display module displays the target image. Other embodiments are described and shown.

### BACKGROUND

### 1. Field of the Invention

The present disclosure relates to an interaction monitoring system, parenting assistance system using the same, and interaction monitoring method using the same. In particular, the present disclosure relates to interaction monitoring system and method, and a parenting assistance system using the same, which provides real-time feedback by monitoring a target interaction during face-to-face interactions.

### 2. Description of Related Art

When conversing with another, giving instructions to another or interacting with another, one cannot recognize or be aware of how he/she is dealing with another. If one can see himself/herself in such situations, there is an advantage in that one can form a better relationship with another.

Particularly, in the context of parenting or childcare, a caregiver's understanding of the mental state of himself/herself or his/her child can play a big role in building a good relationship between the caregiver and child. For example, a situation where a parent unintentionally gets angry should be avoided, because it is difficult for such situation to bring positive effect on child-rearing or parenting. But, there are often times where a caregiver is not aware of the situation in which the caregiver gets angry.

### BRIEF SUMMARY

To solve such problems as above, an object of the present disclosure is to provide an interaction monitoring system, which provides real-time feedback by monitoring a target situation during face-to-face interactions.

Another object of the present disclosure is to provide a parenting assistance system, which uses the interaction monitoring system.

Yet another object of the present disclosure is to provide an interaction monitoring method using the interaction monitoring system.

According to an embodiment of the present disclosure, an interaction monitoring system may comprise an environment collection module, interaction monitoring module, interaction segmentation module, and display module. The environment collection module detects surrounding environment and generates a data stream. The interaction monitoring module generates a feature data stream by extracting feature value of the data stream. The interaction segmentation module determines a target situation, which indicates a user's state or condition, from the feature data stream and generates a target image, which indicates the target situation. The display module displays the target image.

According to an embodiment, the target image may comprise a video stream including the user's face.

According to an embodiment, the environment collection module may comprise an image recording device.

According to an embodiment, the environment collection module may comprise a sound recording device.

According to an embodiment, the environment collection module may be situated on a counterpart that is in interaction with the user.

According to an embodiment, the environment collection module may comprise a skin-resistance detection unit for determining skin resistance of the user.

According to an embodiment, the interaction monitoring module may determine occurrence of conversation between the user and the counterpart.

According to an embodiment, the interaction monitoring module may determine voice volume of the user or the counterpart.

According to an embodiment, the interaction monitoring module may determine the speech rate of the user or the counterpart.

According to an embodiment, the interaction monitoring module may determine the user's eye movement or gaze.

According to an embodiment, the interaction monitoring module may determine the user's face expression.

According to an embodiment, the interaction monitoring module may determine the user's emotional state.

According to an embodiment, the interaction monitoring module may output control signal controlling on/off of a device within the environment collection module to the environment collection module based on occurrence of conversation between the user and the counterpart, or distance between the user and the counterpart.

According to an embodiment, the interaction monitoring module and the environment collection module may determine the distance between the user and the counterpart using wireless communication.

According to an embodiment, the display module may be situated on the counterpart.

According to an embodiment, the display module may be worn or attached near the counterpart's upper body (e.g., chest).

According to an embodiment, the display module may be situated on the user.

According to an embodiment, the display module may be an external device situated away from the user or the counterpart.

According to an embodiment, the display module may replay sound corresponding to the target situation.

According to an embodiment, the display module may display the target image when the target situation occurs and not display the target image when the target situation does not occur.

According to an embodiment, the display module may display the target image when the target situation occurs and display the user's face when the target situation does not occur.

According to an embodiment, the interaction monitoring system may further comprise a segmentation rule storage unit for storing segmentation rule for determining the target situation from the feature data stream and outputting the segmentation rule to the interaction segmentation module.

According to an embodiment, the interaction monitoring system may further comprise a recognition check module for determining whether the user recognizes or checks the display module.

According to an embodiment, the recognition check module may output display control signal controlling operation of the display module to the display module, according to whether the user recognizes and checks the display module.

According to an embodiment, the recognition check module may receive the data stream from the environment collection module and determines whether the user recognizes and checks the display module.

According to an embodiment, the recognition check module may be a face detection unit for determining presence of the user's face.

According to an embodiment, the recognition check module may be a gaze tracking unit for determining gaze vector of the user.

According to an embodiment, when the interaction segmentation module determines the target situation and generates the target image and the recognition check module determines that the user recognizes and checks the display module, the display module may display the target image.

According to an embodiment, the interaction monitoring system may further comprise a second environment collection module for outputting a second data stream to the recognition check module.

According to an embodiment, the interaction monitoring system may further comprise a target image storage unit for receiving and storing the target image from the interaction segmentation module and outputting the target image to the display module upon request for the target image.

According to an embodiment, the target image storage unit may receive information as to whether the user recognizes and check the display module from the recognition check module and store the target image together with the information.

According to another embodiment, the interaction monitoring system may comprise a sensor for detecting surrounding environment and generating a data stream; a mobile device for extracting feature value of the data stream and generating a feature data stream, determining a target situation indicating a user's state or condition from the feature data stream, and generating a target image indicating the target situation; and a display device for displaying the target image.

According to another embodiment, the interaction monitoring system may comprise a first mobile device for detecting surrounding environment and generating a data stream; a second mobile device for extracting feature value of the data stream and generating a feature data stream, determining a target situation indicating a user's state or condition from the feature data stream, and generating a target image indicating the target situation.

According to another embodiment, the first mobile device or the second mobile device may display the target image.

According to an embodiment, a parenting assistance system may comprise the interaction monitoring system, wherein the display module is situated in the first mobile device or the second mobile device.

According to an embodiment, an interaction monitoring method may comprise: detecting surrounding environment and generating a data stream; extracting feature value of the data stream and generating a feature data stream; determining a target situation indicating a user's mental state from the feature data stream; generating a target image indicating the target situation; and displaying the target image.

As above, in the exemplary embodiments of the interaction monitoring system, parenting assistance system, and interaction monitoring method, the system and method generate the target image of the user's target situation, and display the target image in the display module, and enable the user to confirm his/her appearance and behavior while in interaction with the counterpart, through the display module.

For example, the caregiver may check his/her own self (appearance) through the display module during interactions with the child in parenting or child-care situations.

Also, the user may more accurately check his/her appearance during interactions with the counterpart by using a recognition check module, which checks whether the user recognizes and checks (or has checked) the target image

Accordingly, the interaction monitoring system significantly improves the relationship between the user and counterpart. In parenting or childcare situation, the interaction monitoring system may perform the function of parenting assistance or support for the parent(s) to form a better relationship with the child.

### BRIEF DESCRIPTION OF THE DRAWINGS

FIG. 1 shows a block diagram of an interaction monitoring system, according to an embodiment.

FIG. 2 shows a concept diagram of operation of an interaction monitoring module of FIG. 1.

FIG. 3 shows a plan diagram of a display module of FIG. 1, according to an embodiment.

FIG. 4 shows a perspective diagram of a display module of an interaction monitoring system, according to another embodiment.

FIG. 5 shows a perspective diagram of a display module of an interaction monitoring system, according to another embodiment.

FIG. 6 shows a flowchart of an exemplary interaction monitoring method using an interaction monitoring system of FIG. 1, according to an embodiment.

FIG. 7 shows a block diagram of an interaction monitoring system, according to another embodiment.

FIG. 8 shows a block diagram of an interaction monitoring system, according to another embodiment.

FIG. 9 shows a block diagram of a recognition check module of FIG. 8, according to an embodiment.

FIG. 10 shows a block diagram of a recognition check module of FIG. 8, according to another embodiment.

FIG. 11 shows a flowchart of an interaction monitoring method using an interaction monitoring system of FIG. 8, according to another embodiment.

FIG. 12 shows a flowchart of an interaction monitoring method using an interaction monitoring system of FIG. 8, according to another embodiment.

FIG. 13 shows a flowchart of an interaction monitoring method using an interaction monitoring system of FIG. 8, according to another embodiment.

FIG. 14 shows a block diagram of an interaction monitoring system, according to another embodiment.

FIG. 15 shows a block diagram of an interaction monitoring system, according to another embodiment.

FIG. 16 shows a block diagram of an interaction monitoring system, according to another embodiment.

### DETAILED DESCRIPTION

Hereinafter, various embodiments of the present invention are shown and described. Particular embodiments are exemplified herein and are used to describe and convey to a person skilled in the art, particular structural, configurational and/or functional, operational aspects of the invention. The present invention may be altered/modified and embodied in various other forms, and thus, is not limited to any of the embodiments set forth.

The present invention should be interpreted to include all alterations/modifications, substitutes, and equivalents that are within the spirit and technical scope of the present invention.

Terms such as “first,” “second,” “third,” etc. herein may be used to describe various elements and/or parts but the elements and/or parts should not be limited by these terms. These terms are used only to distinguish one element and/or part from another. For instance, a first element may be termed a second element and vice versa, without departing from the spirit and scope of the present invention.

When one element is described as being “joined” or “connected” etc. to another element, the one element may be interpreted as “joined” or “connected” to that another element directly or indirectly via a third element, unless the language clearly specifies. Likewise, such language as “between,” “immediately between,” “neighboring,” “directly neighboring” etc. should be interpreted as such.

Terminology used herein is for the purpose of describing particular exemplary embodiments only and is not intended to limit the present invention. As used herein, singular forms (e.g., “a,” “an”) include the plural forms as well, unless the context clearly indicates otherwise. The language “comprises,” “comprising,” “including,” “having,” etc. are intended to indicate the presence of described features, numbers, steps, operations, elements, and/or components, and should not be interpreted as precluding the presence or addition of one or more of other features, numbers, steps, operations, elements, and/or components, and/or grouping thereof.

Unless otherwise defined, all terms (including technical and scientific terms) used herein have same meaning as those commonly understood by a person with ordinary skill in the art to which this invention pertains. Terms, such as those defined in commonly used dictionaries, should be interpreted as having meaning that is consistent with their meaning in the context of the relevant art and should not be interpreted in an idealized or overly formal sense unless expressly so defined herein.

Hereafter, various embodiments of the present invention are described in more detail with reference to the accompanying drawings. Same reference numerals are used for the same elements in the drawings, and duplicate descriptions are omitted for the same elements or features.

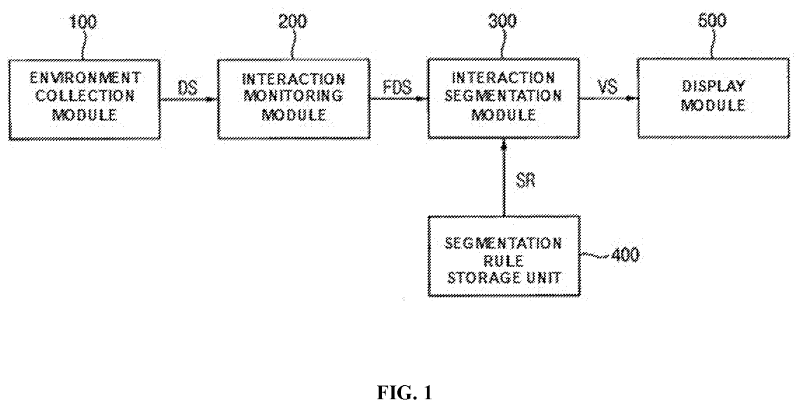

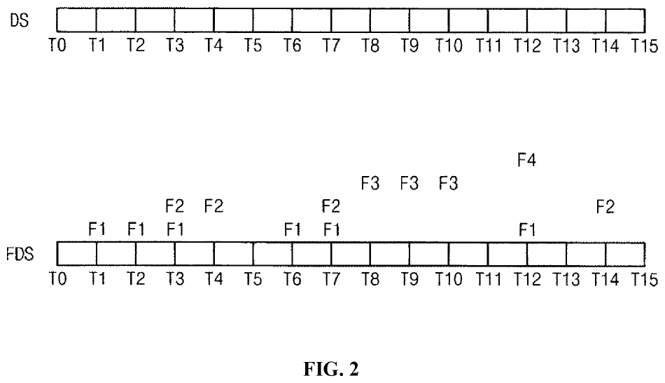

FIG. 1 shows a block diagram of an interaction monitoring system, according to an embodiment. FIG. 2 shows a concept diagram of operation of the interaction monitoring module (200) of FIG. 1.

Referring to FIGS. 1 and 2, the interaction monitoring system may be a system, which monitors interaction(s) of a user and a counterpart (e.g., another person or third party). The interaction monitoring system may detect and capture a target interaction or situation during face-to-face interaction(s) between/among the user and counterpart and provide real-time feedback on/about the target situation to the user. For example, the target situation may and indicate the user's mental or emotional state. And a target image or video displaying and indicating a situation where the user is angry may be generated and provided to the user and enable the user to check his/her own state/status in real time.

The interaction monitoring system may be used as a parenting or childcare assistance system. The interaction monitoring system may detect and capture a target interaction or situation during face-to-face interaction(s) between a parent or caregiver and a child, and generate a target image of or as to the target situation and provide real-time feedback to the caregiver. The target image may be displayed in a display module, and the display module may be placed on the child's body. For example, the display module may be a necklace-type smartphone. The display module may also be attached to the child's clothes.

The interaction monitoring system may comprise an environment collection module (100), interaction monitoring module (200), interaction segmentation module (300), and display module (500).

The environment collection module (100) may detect surrounding environment and generates a data stream (DS).

The environment collection module (100) may comprise an imaging or video device (e.g., camera). The environment collection module (100) may further comprise a recording device (e.g., microphone).

The environment collection module (100) may detect bio- or body signal(s) of the user. For example, the environment collection module (100) may (further) comprise a skin response or resistance detecting device or sensor for determining skin response or resistance of the user. The user's mental or emotional state may be determined based on the user's skin resistance. For example, the environment collection module (100) may (further) comprise a heart-rate detecting device for determining a heart rate of the user. The user's mental or emotional state may be determined based on the user's heart rate.

The environment collection module (100) may detect bio- or body signal(s) of the counterpart (e.g., another person or third party). For example, the environment collection module (100) may (further) comprise a skin resistance detecting device for determining skin resistance of the counterpart. The counterpart's mental or emotional state may be determined based on the counterpart's skin resistance. For example, the environment collection module (100) may (further) comprise a heart-rate detecting device for determining a heart rate of the counterpart. The counterpart's mental or emotional state may be determined based on the counterpart's heart rate. The monitoring system may reference the mental or emotional state of the counterpart to determine the target interaction or situation.

The environment collection module (100) may be placed on the user's body's body. The environment collection module (100) may be placed on the counterpart's body. The environment collection module (100) may be an external device or apparatus, which is placed or arranged away from the user or counterpart.

For example, a part or portion the environment collection module (100) may be arranged on the counterpart's body; and a part or portion of the environment collection module (100) may be arranged on the user's body.

For example, a part or portion the environment collection module (100) may be arranged on the counterpart's body; and a part or portion of the environment collection module (100) may the external device or apparatus.

For example, a part or portion the environment collection module (100) may be arranged on the counterpart's body; and a part or portion of the environment collection module (100) may be arranged on the user's body; and a part or portion of the environment collection module (100) may the external device or apparatus.

The interaction monitoring module (200) may extract a feature value of the data stream (DS) and generates a feature data stream (FDS).

For example, the interaction monitoring module (200) may determine an occurrence of communication or conversation between the user and counterpart, voice level (e.g., volume) of the user or counterpart, and speed or rate of the user or counterpart's speech.

For example, the interaction monitoring module (200) may determine the user's line of sight (gaze; eye movement, direction, etc.) and (facial) expression.

For example, the interaction monitoring module (200) may determine the user's mental or emotional state, such as the user's level(s) of stress, pleasure, anger, etc.

The interaction monitoring module (200) may determine verbal cue (e.g., semantics) and non-verbal cue (pitch, speech rate, turn-taking).

The interaction monitoring module (200) may determine content of the user's speech. The interaction monitoring module (200) may determine the user's pitch, speech rate, and speaking turn or turn-taking (between the user and counterpart).

An input of the interaction monitoring module (200) may be the data stream (DS), and an output of the interaction monitoring module (200) may be a/the feature data stream (FDS) in which a/the feature value is tagged on the data stream (DS).

Referring to FIG. 2, the data stream (DS) and feature data stream (FDS) are represented on time frame from timepoints 0 to 15th as (T1, T2, T3, T4, T5, T6, T7, T8, T9, T10, T11, T12, T13, T14, T15).

For example, the interaction monitoring module (200) may extract a 1st feature value (F1) at the 1st timepoint (T1), 2nd timepoint (T2) and 3rd timepoint (T3), and tag the 1st feature value (F1) at the 1st timepoint (T1), 2nd timepoint (T2), and 3rd timepoint (T3) of the data stream (DS), and generate the first feature data stream (FDS).

The interaction monitoring module (200) may extract a 2nd feature value (F2) at the 3rd timepoint (T3) and 4th timepoint (T4), and tag the 2nd feature value (F2) at the 3rd timepoint (T3) and 4th timepoint (T4) of the data stream (DS), and generate the first feature data stream (FDS).

At the 3rd timepoint (T3), the 1st feature value (F1) and 2nd feature value (F2) may both be tagged thereat.

At the 6th timepoint (T6), the first feature value (F1) may be extracted and tagged with the 1st feature value (F1), and at the 7th timepoint (T7), the 1st feature value (F1) and 2nd feature value (F2) may be extracted and tagged. From the 8th timepoint (T8) through 10th timepoint (T10), the 3rd feature value (F3) may be extracted and tagged. At the 11th timepoint (T11), the 1st feature value (F1) and 4th feature value (F4) may be extracted and tagged, and at the 14th timepoint (T14), the 2nd feature value (F2) may by extracted and tagged.

The feature value(s) may be tagged via on/off method, or with a specific value. For example, the feature value(s) may be tagged via on/off method when the feature value(s) is/are occurrence of speech (e.g., whether or not a person is speaking), or with a s specific value when the feature value(s) is/are volume (loudness) of the user's voice.

The interaction segmentation module (300) may receive the feature data stream (FDS) from the interaction monitoring module (200). The interaction segmentation module (300) may determine a target interaction or situation, which indicate the user's particular state or condition, from the feature data stream (FDS), and generate a target image or video stream (VS) showing the target situation.

The target situation may for instance, be a situation where the user is angry, or is laughing, or is in a fight with another person.

The interaction monitoring system may store a segmentation rule (SR) to determine the target situation from the feature data stream (FDS), and may further comprise a segmentation rule storage unit (400) to output the segmentation rule (SR) to the interaction segmentation module (300).

For example, the interaction segmentation module (300) may receive the feature data stream (FDS) and generate the target image (VS) according to the segmentation rule (SR).

The target image (VS) may be a video. The target image (VS) may be a moving image, which includes the user's face. Different from this, the target image (VS) may be a still/static image. The target image (VS) may be a captured image.

The target image (VS) may also be a modified or composite image or video based on the user's state or condition. For example, the target image (VS) may be an image in which a filter is applied to an image of the user's face or an image in which a picture, appearance or particular image are added on or composited: e.g., the target image (VS) may be an image in which the user's face is imposed on or reflected onto a (certain) character.

For example, the segmentation rule (SR) may be (counterpart's gaze (stare) index>0.7 & user anger index>0.8), and when the segmentation rule (SR) is satisfied, the target image (VS) may be a set-length video stream, which includes a situation in which the user is watching the counterpart with a scary face.

For example, the interaction segmentation module (300) may determine as a situation where the user gets angry, a section in which the user's speech gets faster and/or the user's stress is higher than a threshold value, and segment a video stream corresponding to the section in which the user is getting angry.

The display module (500) may display the target image (VS).

For example, the display module (500) may display the target image (VS) when the target situation does not occur; and vice versa.

For example, the display module (500) may display the target image (VS) when the target image (VS) occurs, and display the user's face when the target image (VS) does not occur. The display module (500) may acquire an image corresponding to the user's face, which is collected by the environment collection module (100), and continuously display the user's face image when the target situation does not occur. The user may check his/her own face as displayed in the display module (500), and receive assistance in controlling his/her emotions in face-to-face interactions.

The display module (500) may (re)play a sound applicable to the target situation.

When a plurality of the target image (VS) is overlappingly generated, the target images may be sequentially displayed in the display module (500). Different from this, when the plurality of the target images is overlappingly generated, most recent of the target images may be displayed in the display module (500). Different yet, in such case, most important of the target images may be displayed in the display module (500).

The display module (500) may be a device, same or analogous to the environment collection module (100); the display module (500) may have functionality overlapping with that of the environment collection module (100).

Differently, the display module (500) may be an independent device from the environment collection module (100).

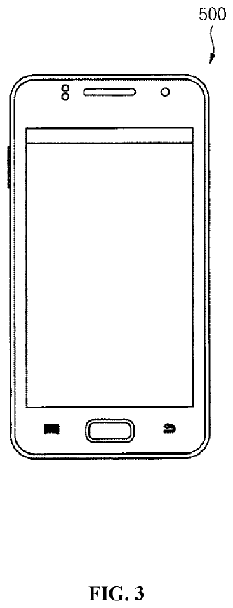

FIG. 3 shows a plan diagram of the display module (500) of FIG. 1, according to an embodiment.

Referring to FIG. 1 to FIG. 3, the display module (500) may be arranged on the counterpart who is interacting with the user. For example, the display module (500) may be attached or worn on the counterpart's chest portion (not shown).

As shown in FIG. 3, the display module (500) may for instance, be a smartphone (e.g., a necklace-type smartphone worn by the counterpart or a smartphone attached to the counterpart's clothes; not shown).

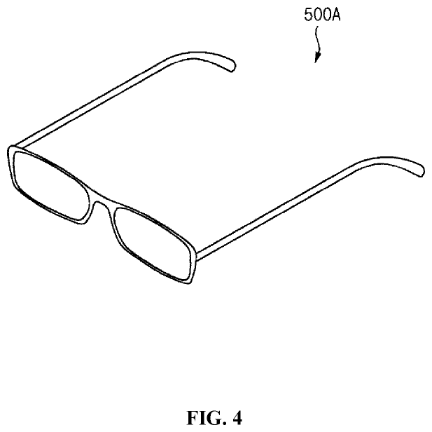

FIG. 4 shows a perspective diagram of the display module (500A) of the interaction monitoring system, according to another embodiment.

The interaction monitoring system according to present embodiment is practically the same with the interaction monitoring system of FIG. 1 and FIG. 3, except for the element of the display module, and the same reference numerals are used to refer to the same or analogous elements, with duplicate description omitted.

Referring to FIG. 1, (FIG. 2) and FIG. 4, the interaction monitoring system may comprise an environment collection module (100), interaction monitoring module (200), interaction segmentation module (300), and a display module (500A).

As shown in FIG. 4, the display module (500A) may for instance, be eyeglasses worn by the user (e.g., virtual reality eyeglasses).

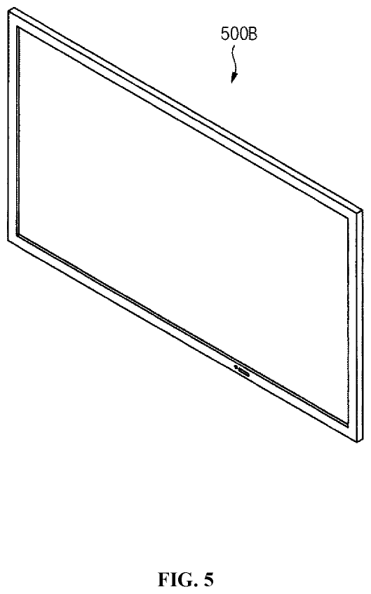

FIG. 5 shows a perspective diagram of the display module (500B) of the interaction monitoring system, according to another embodiment.

The interaction monitoring system according to present embodiment is practically the same with the interaction monitoring system of FIG. 1 and FIG. 3, except for the element of the display module, and the same reference numerals are used to refer to the same or analogous elements, with duplicate description omitted.

Referring to FIG. 1, (FIG. 2) and FIG. 5, the interaction monitoring system may comprise an environment collection module (100), interaction monitoring module (200), interaction segmentation module (300), and a display module (500B).

As shown in FIG. 5, the display module (500B) may for instance, be a wall-type television installed in the user's and/or counterpart's interaction environment. Differently, the display module (500B) may also be a stand-type television, computer monitor, notebook PC etc.

The display module (500B) may be an external display device, which is installed or disposed away from the user and/or counterpart whom the user is interacting with.

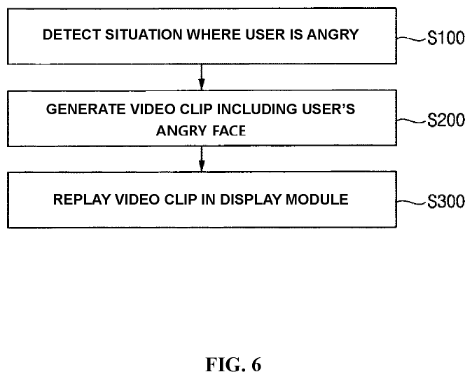

FIG. 6 shows a flowchart of an exemplary interaction monitoring method using the interaction monitoring system of FIG. 1, according to an embodiment.

Referring to FIG. 1 and FIG. 6, the interaction monitoring module (200) extracts (a) feature value(s) from data stream (DS) generated by the environment collection module (100) to generate feature data stream (FDS).

The interaction segmentation module (300) may determine the target situation from the feature data stream (FDS) to generate the target image (VS), which includes the target situation.

Suppose the target situation is a situation where the user is angry, the interaction segmentation module (300) may detect a situation where the user is angry from the feature data stream (FDS) (Step S100).

The interaction segmentation module (300) may generate the target image (e.g., “video clip”), which includes the user's angry face (Step S200).

The display module (500) may (re)play the target image (e.g., “video clip”), which includes the user's angry face.

In one embodiment, the environment collection module (100) may be a sensor that generates the data stream (DS) by detecting surrounding environment. The interaction monitoring module (200) and interaction segmentation module (300) may be the user's mobile device. That is, the user's mobile device may extract the feature values of the data stream (DS) and generate the feature data stream (FDS), and determine the target situation, which indicates the user's state or condition, from the feature data stream (FDS), and generate the target image (VS), which shows the target situation. The display device may display the target image. The display device may be configured as a separate device from the user's mobile device.

In another embodiment, a 1st mobile device may detect the surrounding environment and generate the data stream (DS). A 2nd mobile deice may extract the feature values of the data stream (DS) and generate the feature data stream (FDS), and determine the target situation, which indicates the user's state or condition, from the feature data stream (FDS), and generate the target image (VS), which shows the target situation. The 1st mobile device may (then) display the target image (VS).

In another embodiment, the interaction monitoring system may be used as a parenting or childcare assistance system. The parenting assistance system may comprise a 1st electronic device in possession of a caregiver and a 2nd electronic device disposed on a child (i.e., person cared for)'s body. The 1st electronic device may determine the target situation showing the caregiver's state or condition based on sensing data, and generate the target image (VS) showing the target situation. The 2nd electronic device may display the target image (VS).

In present embodiment, the target situation may be a situation, which provides assistance in parenting or childcare, and for instance, include a situation where the user is angry, the user is laughing, or the user is in fight with another person, etc.

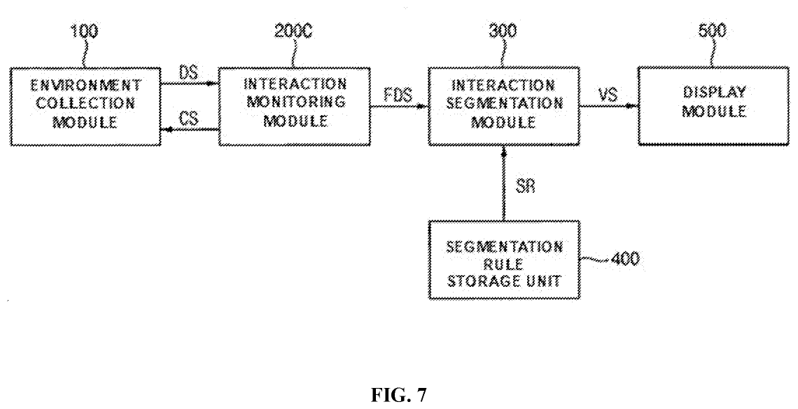

FIG. 7 shows a block diagram of the interaction monitoring system, according to another embodiment.

The interaction monitoring system according to present embodiment is practically the same with the interaction monitoring system of FIG. 1 and FIG. 6, except for the elements of the environment collection module and the interaction monitoring module, and the same reference numerals are used to refer to the same or analogous elements, with duplicate description omitted.

Referring to FIG. 2 to FIG. 7, the interaction monitoring system may comprise an environment collection module (100), interaction monitoring module (200C), interaction segmentation module (300), and display module (500).

In present embodiment, the interaction monitoring module (200C) may output to the environment collection module (100), control signal (CS), which controls on/off of device (d) within the environment collection module (100), based on occurrence of face-to-face interaction between the user and counterpart or on a distance of the user and the counterpart.

For example, when the user and counterpart are not in a face-to-face situation, the interaction monitoring system may not be required to operate. Also, when the user and the counterpart are more than a certain distance apart, the interaction monitor system may not be required to operate. Thus, in this case, power consumption of the interaction monitoring system is reduced by preventing the environment collection module (100) from operating.

For example, the interaction monitoring module (200C) may determine whether a face-to-face interaction occurs between the user and counterpart through the data stream (DS) received from the environment collection module (100).

For example, the interaction monitoring module (200C) may determine the distance between the user and the counterpart through the data stream (DS) received from the environment collection module (100).

For example, the interaction monitoring module (200C) and the environment collection module (100) may determine the distance between the user and the counterpart via wireless communication. Here, the interaction monitoring module (200C) may be a device in possession by the user, and the environment collection module (100) may be a device in possession by the counterpart.

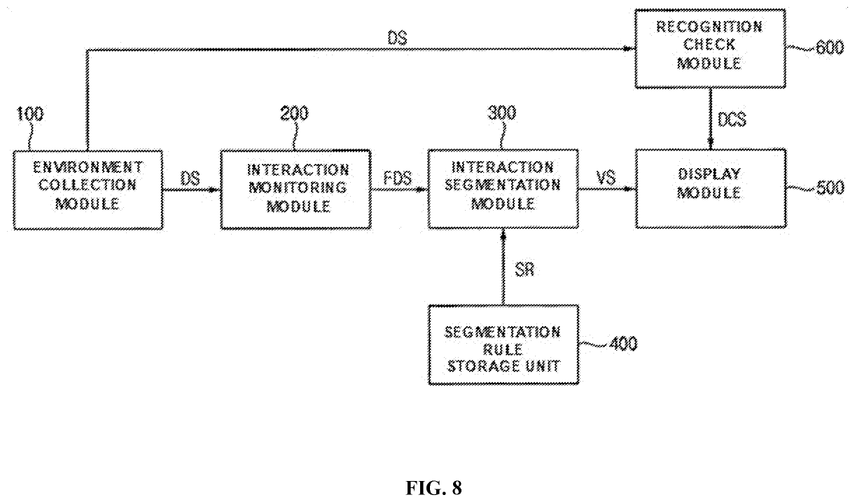

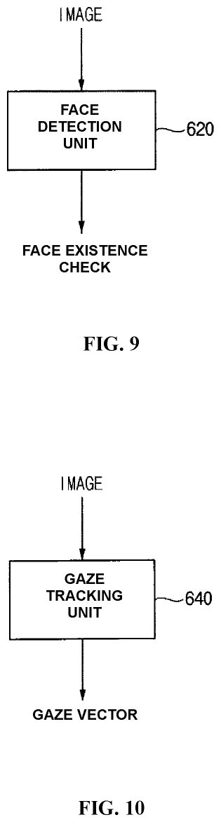

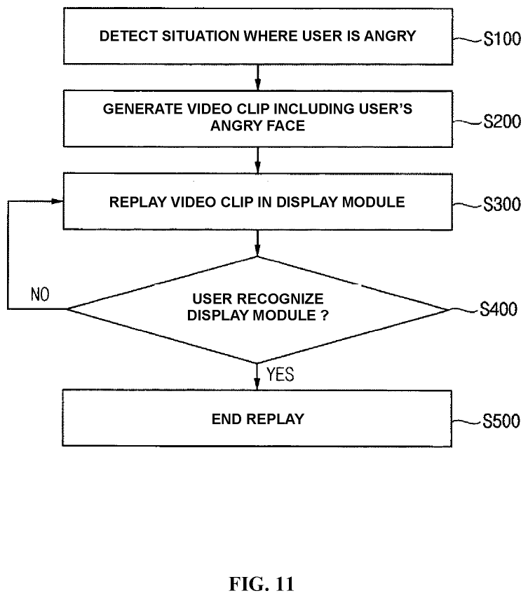

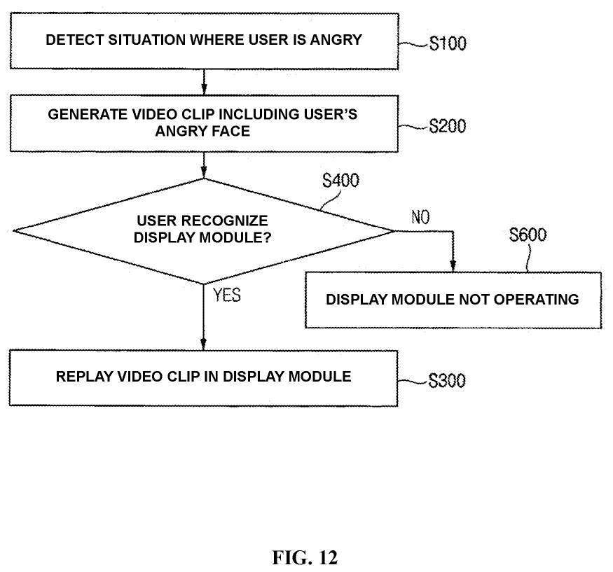

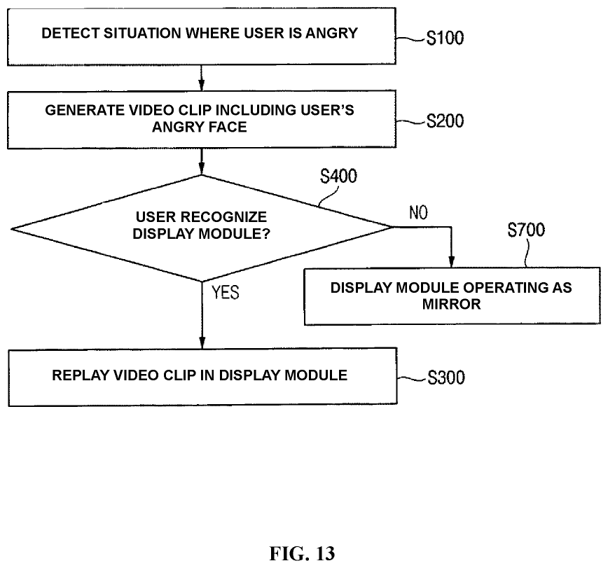

FIG. 8 shows a block diagram of the interaction monitoring system, according to another embodiment. FIG. 9 shows a block diagram of the recognition check module of FIG. 8, according to an embodiment. FIG. 10 shows a block diagram of the recognition check module of FIG. 8, according to another embodiment. FIG. 11 shows a flowchart of an interaction monitoring method using an interaction monitoring system of FIG. 8, according to another embodiment. FIG. 12 shows a flowchart of the interaction monitoring method using the interaction monitoring system of FIG. 8, according to another embodiment. FIG. 13 shows a flowchart of the interaction monitoring method using the interaction monitoring system of FIG. 8, according to another embodiment.

The interaction monitoring system according to present embodiment is practically the same with the interaction monitoring system of FIG. 1 and FIG. 6, except for the further comprised element of a recognition check module, and the same reference numerals are used to refer to the same or analogous elements, with duplicate description omitted.

Referring to FIG. 2 to FIG. 6, and FIG. 8 to FIG. 13, the interaction monitoring system (100) may comprise an environment collection module (100), interaction monitoring module (200C), interaction segmentation module (300), and display module (500).

In present embodiment, the interaction monitoring system may further comprise a recognition check module (600), which checks whether or not the user recognizes (i.e., has recognized or acknowledged) the display module (500).

The recognition check module (600) may output to the display module (500), display control signal (DCS), which controls operation of the display module, based on whether or not the user recognizes and checks the display module (500).

In present embodiment, the recognition check module (600) may receive the data stream (DS) from the environment collection module (100) and check whether the user recognizes and checks the display module (500).

As shown in FIG. 9, the recognition confirmation module (600) may be a face detection unit (620) which determines a presence or existence of the user's face. The face detection unit (620) may receive input image (IMAGE) from the environment collection module (100) and determine whether or not the user's face is present or exists in the input image (IMAGE). Here, the environment collection module (100), which generates the input image (IMAGE) and transmits the input image (IMAGE) to the face detection unit (620), may be disposed or arranged in the display module (500).

As shown in FIG. 10, the recognition check module (600) may be a gaze tracking device (640) which determines the user's gaze vector or eye movement. The gaze tracking unit (640) may receive the input image (IMAGE) from the environment collection module (100) and determine the user's gaze vector. Here, the environment collection module (100), which generates the input image (IMAGE) and transmits the input image (IMAGE) to the gaze tracking unit (640) may be disposed or arranged either within or without (e.g., outside) the display module (500).

Referring to FIG. 11, supposing that the target situation is a situation where the user is angry, the interaction segmentation module (300) may detect a/the situation where the user is angry from the feature data stream (FDS) (Step S100).

The interaction segmentation module (300) may generate the target image (e.g., VS, video clip) including the user's angry face (Step S200).

The display module (500) may instantly (re)play the target image (e.g., video clip) including the user's angry face (Step S300). Here, the recognition check module (600) may determine whether or the user recognizes and checks the display module (500).

When the recognition check module (600) determines that the user recognizes and checks (or has checked) the display module (500) for more than a given time while the target image (e.g., video clip) is displaying in the display module (500) (Step S400), the display module (500) may end the display (e.g., “replay”) of the target image (e.g., video clip).

When the recognition check module (600) determines that the user does not recognize and check (or has not checked) the display module (500) for more than a given time while the target image (e.g., video clip) is displaying in the display module (500), the display module (500) may continuously or repeatedly display the target image (e.g., video clip). That is, the recognition confirmation module 600 may be used to check whether the user checks (or has checked) the target image (e.g., video clip) as to the target situation. When the recognition check module (600) determines to the contrary, the display (e.g., “replay”) may end.

After the display of the target image ends, the display module (500) may or may not display any image or video, and may display the user's face in real-time.

Referring to FIG. 12, supposing that the target situation is a situation where the user is angry, the interaction segmentation module (300) may detect a/the situation where the user is angry from the feature data stream (FDS) (Step S100).

The interaction segmentation module (300) may generate the target image (e.g., video clip), which includes the user's angry face (Step S200).

In present embodiment, when the recognition check module (600) determines that the user checks (or has checked) the display module (500) (Step S400), the display module (500) may start displaying the target image (e.g., video clip) (Step S300). That is, when the user does not see the display module (500), the display module (500) may not display the target image (e.g., video clip) (Step S600); but when the user sees the display module (500), the display module (500) may then display the target image (e.g., the video clip) (Step S300).

In the case of FIG. 12, as was described in FIG. 11, when the recognition check module (600) determines that the user does not recognize and check (or has not checked) the display module (500) for more than a given time while the target image (e.g., video clip) is displaying in the display module (500), the display module (500) may end displaying the target image (e.g., video clip). After the displaying of the target image ends, the display module (500) may or may not display any image or video.

Referring to FIG. 13, supposing that the target situation is a situation where the user is angry, the interaction segmentation module (300) may detect a/the situation where the user is angry from the feature data stream (FDS) (Step S100).

The interaction segmentation module (300) may generate the target image (e.g., video clip), which includes the user's angry face (Step S200).

In present embodiment, when the recognition check module (600) determines that the user checks (or has checked) the display module (500) (Step S400), the display module (500) may start displaying the target image (e.g., video clip) (Step S300). That is, when the user does not see the display module (500), the display module (500) may continuously or repeatedly display the user's face (e.g., as a default state) in real-time (Step S700); but when the user sees the display module (500), the display module (500) may then display the target image (e.g., the video clip) (Step S300).

In the case of FIG. 13, as was described in FIG. 11, when the recognition check module (600) determines that the user does not recognize and check (or has not checked) the display module (500) for more than a given time while the target image (e.g., video clip) is displaying in the display module (500), the display module (500) may end displaying the target image (e.g., video clip). After the displaying of the target image ends, the display module (500) may continuously or repeatedly display the user's face in real-time.

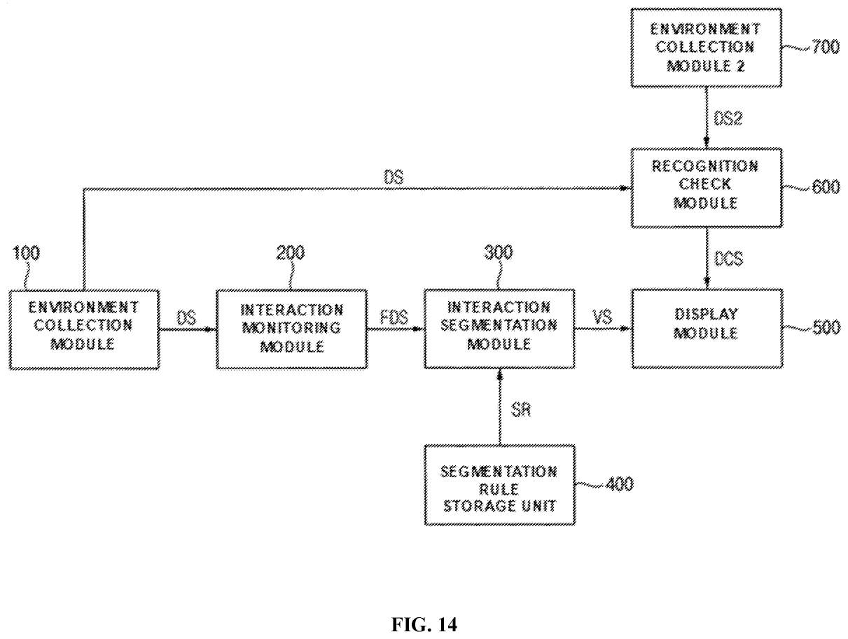

FIG. 14 shows a block diagram of the interaction monitoring system, according to another embodiment.

The interaction monitoring system according to present embodiment is practically the same with the interaction monitoring system of FIG. 8 and FIG. 13, except for the further comprised element of a 2nd environment collection module, and the same reference numerals are used to refer to the same or analogous elements, with duplicate description omitted.

Referencing FIG. 2 to FIG. 6 and FIG. 9 to FIG. 14, the interaction monitoring system may comprise an environment collection module (100), interaction monitoring module (200), interaction segmentation module (300), and display module (500).

In present embodiment, the interaction monitoring system may further comprise a recognition check module (600), which checks whether or not the user recognizes (i.e., has recognized or acknowledged) the display module (500).

The recognition check module (600) may output to the display module (500), display control signal (DCS), which controls operation of the display module, based on whether or not the user recognizes and checks the display module (500).

In present embodiment, the recognition check module (600) may further comprise a 2nd environment collection module (700), which outputs a 2nd data stream (DS2) to the recognition check module (600).

The recognition check module (600) may receive the 2nd data stream (DS2) from the 2nd environment collection module (700) and check whether the user recognizes and checks the display module (500).

The 2nd data stream (DS2) at or needed by the recognition check module (600) may be different from the data stream (DS1) at or need by the interaction monitoring module (200). Thus, the interaction monitoring system may further comprise the 2nd environment collection module (700), which outputs the 2nd data stream (DS2) to the recognition check module (600).

In the case of FIG. 14, the recognition check module (600) receives the data stream (DS) from the environment collection module (100), and additionally receive the 2nd data stream (DS2) from the 2nd environment collection module (700). Differently, the recognition check module (600) may also not receive the data stream (DS) from the environment collection module (100) but receive only the 2nd data stream (DS2) from the 2nd environment collection module (700).

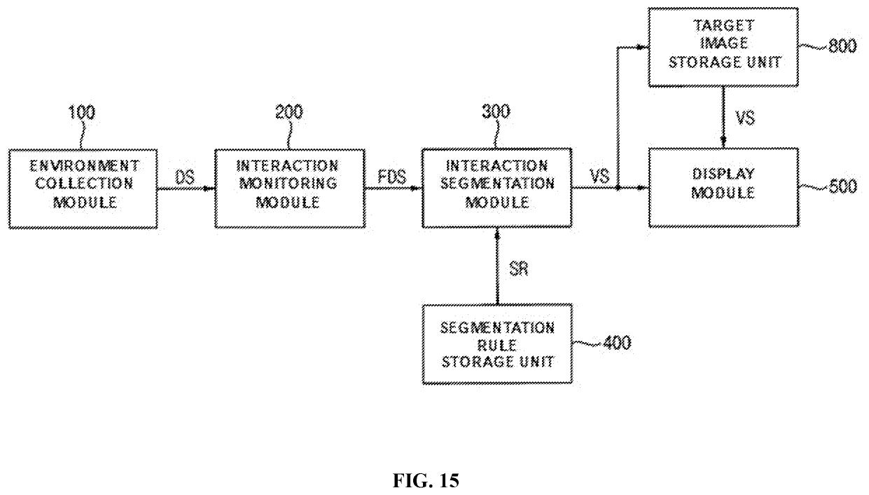

FIG. 15 shows a block diagram of the interaction monitoring system, according to another embodiment.

The interaction monitoring system according to present embodiment is practically the same with the interaction monitoring system of FIG. 1 and FIG. 6, except for the further comprised element of a target image storage unit, and the same reference numerals are used to refer to the same or analogous elements, with duplicate description omitted.

Referencing FIG. 2 to FIG. 6 and FIG. 15, the interaction monitoring system may comprise an environment collection module (100), interaction monitoring module (200), interaction segmentation module (300), and display module (500).

In present embodiment, the interaction monitoring system may further comprise a target image storage unit (800), which receives the target image (VS) from the interaction segmentation module (300) and stores the target image (VS), and outputs the target image (VS) to the display module (500) upon request for the target image (VS).

In present embodiment, the target image (VS) as to the target situation may be stored in the target image storage unit (800) and the target image (VS), (re)played when the user requests.

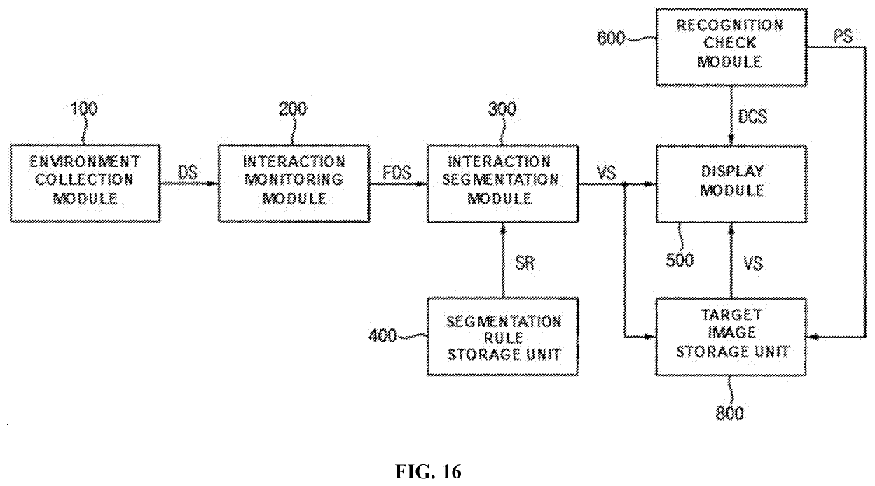

FIG. 16 shows a block diagram of the interaction monitoring system, according to another embodiment.

The interaction monitoring system according to present embodiment is practically the same with the interaction monitoring system of FIG. 8 and FIG. 13, except for the further comprised element of a target image storage unit, and the same reference numerals are used to refer to the same or analogous elements, with duplicate description omitted.

Referencing FIG. 2 to FIG. 6 and FIG. 13 and FIG. 15, the interaction monitoring system may comprise an environment collection module (100), interaction monitoring module (200), interaction segmentation module (300), and display module (500).

In present embodiment, the interaction monitoring system may further comprise a recognition check module (600), which checks whether or not the user recognizes (i.e., has recognized or acknowledged) the display module (500).

The recognition check module (600) may output to the display module (500), display control signal (DCS), which controls operation of the display module, based on whether or not the user recognizes and checks the display module (500).

In present embodiment, the interaction monitoring system may further comprise a target image storage unit (800), which receives the target image (VS) from the interaction segmentation module (300) and stores the target image (VS), and outputs the target image (VS) to the display module (500) upon request for the target image (VS).

In present embodiment, the target image storage unit (800) may receive the user's recognition/check status for the display module (500) from the recognition check module (600) and store with the target image (VS) and the user's recognition/check status for the display module (500).

In present embodiment, the target image (VS) as to the target situation may be stored in the target image storage unit (800) and the target image (VS), (re)played when the user requests. The target image storage unit (800) may store the target image (VS) along with the user's recognition/check status for the display module (500), and as such, the target image (VS) that the user has not recognized or checked may be (re)played again upon the user's request.

According to present embodiment, the target image (VS) may be generated for the target situation during interaction between the user and the counterpart, and the target image (VS), displayed by the display module (500) to enable the user to check his/her appearance through the display module (500) while associating and responding to the counterpart during the interaction.

For example, in parenting or childcare situation, a caregiver may check his/her appearance during interactions with a child through the recognition check module (500).

Also, using the recognition check module (600) to check whether the target image (VS) being displayed in the display module (500) is recognized by the user, the user is able to more accurately check, review, and confirm his/her appearance during interactions with the counterpart.

Accordingly, using the interaction monitoring system and method enables the user and counterpart (another person or third party) to build a better relationship with each other. In parenting or childcare situation, the interaction monitoring system performs the function of parenting assistance or support for the parent(s) to form a better relationship with the child.

According to the present disclosure, real-time feedback may be provided by monitoring a target situation during face-to-face interaction.

Exemplary embodiments have been described in detail with references to the accompanying drawings, for illustrative purposes (and) to solve technical problems. Although the description above contains much specificity, these should not be construed as limiting the scope of the exemplary embodiments. The exemplary embodiments may be modified and implemented in various forms and should not be interpreted as thus limited. A person skilled in the art will understand that various modifications and alterations may be made without departing from the spirit and scope of the description and that such modifications and alterations are within the scope of the accompanying claims.

### REFERENCE NUMERALS

100: Environment Collection Module
200, 200C: Interaction Monitoring Module
300: Interaction Segmentation Module
400: Segmentation Rule Storage Unit
500, 500A, 500B: Display Module
600: Recognition Check Module
620: Face Detection Unit 640: Gaze Tracking Unit
700: 2nd Environment Collection Module
800: Target Image Storage Unit

What is claimed is:

1. An interaction monitoring system comprising:
an environment collection module for detecting surrounding environment and generating a data stream,
an interaction monitoring module for extracting feature value of the data stream and generating a feature data stream,
an interaction segmentation module for determining a target situation indicating a user's state or condition from the feature data stream, and generating a target image showing the target situation,
a display module for displaying the target image, and
a recognition check module for determining whether the user recognizes and checks the display module.

2. The interaction monitoring system according to claim 1, wherein the environment collection module is situated on a counterpart that is in interaction with the user.

3. The interaction monitoring system according to claim 1, wherein the environment collection module comprises an image recording device.

4. The interaction monitoring system according to claim 3, wherein the environment collection module comprises a skin-resistance detection unit for determining skin resistance of the user.

5. The interaction monitoring system according to claim 3, wherein the environment collection module comprises a heart rate detection unit for determining heart rate of the user.

6. The interaction monitoring system according to claim 1, wherein the interaction monitoring module determines occurrence of conversation between the user and a counterpart, or voice volume of the user or the counterpart, or the speech rate of the user or the counterpart.

7. The interaction monitoring system according to claim 1, wherein the interaction monitoring module determines the user's eye movement or gaze and face expression.

8. The interaction monitoring system according to claim 1, wherein the interaction monitoring module determines the user's emotional state.

9. The interaction monitoring system according to claim 1, wherein the interaction monitoring module outputs control signal controlling on/off of a device within the environment collection module to the environment collection module based on occurrence of conversation between the user and a counterpart, or distance between the user and the counterpart.

10. The interaction monitoring system according to claim 9, wherein the interaction monitoring module and the environment collection module determine the distance between the user and the counterpart using wireless communication.

11. The interaction monitoring system according to claim 1, wherein the interaction monitoring system further comprises
a segmentation rule storage unit for storing segmentation rule for determining the target situation from the feature data stream and outputting the segmentation rule to the interaction segmentation module.

12. The interaction monitoring system according to claim 1, wherein the target image comprises a video stream including the user's face.

13. The interaction monitoring system according to claim 1, wherein the display module:
displays the target image when the target situation occurs, and
does not display the target image or displays the user's face when the target situation does not occur.

14. The interaction monitoring system according to claim 1, wherein the display module is situated on a counterpart that is in interaction with the user.

15. The interaction monitoring system according to claim 1, wherein the display module is situated on the user.

16. The interaction monitoring system according to claim 1, wherein the display module is an external device situated away from the user or a counterpart that is in interaction with the user.

17. The interaction monitoring system according to claim 1, wherein the display module replays sound corresponding to the target situation.

18. The interaction monitoring system according to claim 1, wherein the recognition check module outputs display control signal controlling operation of the display module to the display module, according to whether the user recognizes and checks the display module.

19. The interaction monitoring system according to claim 18, wherein the recognition check module receives the data stream from the environment collection module and determines whether the user recognizes and checks the display module.

20. The interaction monitoring system according to claim 18, wherein the recognition check module is a face detection unit for determining presence of the user's face.

21. The interaction monitoring system according to claim 18, wherein the recognition check module is a gaze tracking unit for determining gaze vector of the user.

22. The interaction monitoring system according to claim 18, wherein:
when the interaction segmentation module determines the target situation and generates the target image and the recognition check module determines that the user recognizes and checks the display module, the display module displays the target image.

23. The interaction monitoring system according to claim 18, wherein the interaction monitoring system further comprises
a second environment collection module for outputting a second data stream to the recognition check module.

24. The interaction monitoring system according to claim 18, wherein the interaction monitoring system further comprises
a target image storage unit for receiving and storing the target image from the interaction segmentation module and outputting the target image to the display module upon request for the target image.

25. The interaction monitoring system according to claim 24, wherein the target image storage unit receives information as to whether the user recognizes and checks the display module from the recognition check module and stores the target image together with the information.

26. The interaction monitoring system according to claim 1, wherein the interaction monitoring system further comprises
a target image storage unit for receiving and storing the target image from the interaction segmentation module and outputting the target image to the display module upon request for the target image.

27. An interaction monitoring system, comprising:
a first mobile device for detecting surrounding environment and generating a data stream;
a second mobile device for
extracting feature value of the data stream and generating a feature data stream, determining a target situation indicating a user's state or condition from the feature data stream, and
generating a target image indicating the target situation; and
a display unit for displaying the target image.

28. A parenting assistance system comprising the interaction monitoring system according to claim 27, wherein the display unit is situated in the first mobile device or the second mobile device.

29. An interaction monitoring method, comprising:
detecting surrounding environment and generating a data stream;
extracting feature value of the data stream and generating a feature data stream;
determining a target situation indicating a user's mental state from the feature data stream;
generating a target image indicating the target situation; and
displaying the target image.
